(19) United States
(12) Patent Application Publication
(10) Pub. No.: US 2026/0257374 A1
(43) Pub. Date: Sep. 3, 2026

(54) ROBOTIC THUMB AND RESPECTIVE CONTROL METHOD

(71) Applicant: BionIT Labs S.r.l., Soleto (LE) (IT)

(72) Inventors: Giovanni Antonio Zappatore, Lecce (IT); Alberto Longo, Melendugno (LE) (IT); Mauro Dimastrogiovanni, San Cesariodi Lecce(LE) (IT); Domenico Tinella, Galatina(LE) (IT); Andrea Accogli, [Lecce(LE) (IT)

(73) Assignee: BionIT Labs S.r.l., Soleto (LE) (IT)

(21) Appl. No.: 19/472,867

(22) PCT Filed: May 3, 2024
(86) PCT No.: PCT/IB2024/054323

§ 371 Date: Oct. 6, 2025

(30) Foreign Application Priority Data
May 12, 2023 (IT) 102023000009585

(51) Int. Cl. B25J 15/00; A61F 2/58

(52) U.S. Cl. CPC B25J 15/0009; A61F 2/586; A61F 2002/587

(57) ABSTRACT

The invention relates to an anthropomorphic thumb for a robotic hand with high strength and compliance characterized by active flexion and controllable rotation both actively and passively controlled by the user.

The robotic hand having four aligned fingers and a relative actuation mechanism to make them rotate with respect to a frame; having a metacarpus fastened to the frame so to be able to rotate and translate along the axis of a cylindrical joint; a thumb fastened to the metacarpus so to be able to rotate along the axis of a rotoidal joint

### BACKGROUND OF THE INVENTION

### 1. Field of the Invention

The present patent application for industrial invention relates to a thumb for a robotic hand with high strength and compliance, characterized by active flexion and controllable rotation both actively and passively by the user.

### 2. Brief Description of the Prior Art

In many robotic hands known at the state of the art, the thumb is moved by means of the same motor controlling the other fingers. Generally, in these cases, there is a first differential stage, whose two outputs are connected to the thumb and the other fingers, respectively. Anyway, this embodiment limits somehow the movement of the thumb to the other fingers' one and does not allow to obtain all the movement configurations of interest. For example, it is not possible to open the thumb while closing the other fingers or vice versa. In other embodiments, the thumb is moved by a dedicated motor, and so it refers to a mechanism kinematically independent with respect to the one of the four aligned fingers.

In any case, the mechanism of the thumb should be able: to allow a rotation of the metacarpus so to move the thumb closer to or away from the metacarpus, and to allow the opening and closing of the two phalanges of the thumb. It is also needed to control the thumb coordinately with the four aligned fingers, both for position and for closing and opening speed, to provide all the various kinds of grasps commonly performed by the hands of a human being.

A first solution to this problem of control is provided in the document EP2125091, wherein an anthropomorphic hand is described, provided with EMG sensors and a sensor to measure the angle between thumb and hand palm. The device described in EP2125091 is configured to move the fingers of the hand as a function of the signals detected by the EMG devices and the signal emitted by the sensor that measures the angle between thumb and hand palm, blocking the movement of one or more fingers to obtain the desired configuration.

Yet, it remains unsolved the problem to provide a thumb for a robotic hand that overcomes the limits linked to what known at the state of the art, and that in particular allows to carry out the movement of rotation both actively, by means of a motor controlled as a function of the signal of sensors detecting user physiological parameters, and passively, by means of the contralateral limb of the user himself, and that, at the same time, is robust and compact and allows a simple replacement, for example in case of failure.

Another technical problem, yet unsolved at the state of the art, is to provide a robotic thumb that the patient can control easily for all the actions to be carried out, without the need to select different grasps by means of complex muscle contractions.

Yet, it remains unsolved the technical problem to provide a method for control ling a robotic hand that allows to realize easily and immediately for the user all the possible grasps performed by a human hand, as a function of the physiological signals (EMG) detected on the user and of the position of the hand itself.

More in particular, it remains unsolved the problem to provide a robotic thumb that reaches the just described aims and that is also highly resistant to mechanical stresses and to the penetration of liquids and powders, so that the need of maintenance interventions during the useful life of the device is reduced.

Yet, it remains unsolved the problem to provide a prosthetic hand whose elastic elements adjustment can be carried out without disassembling the device. Actually, this makes it impossible with the known devices—that the orthopedic technician can carry out a fine setup to adapt its functioning to the preferences of each single user.

### SUMMARY OF THE INVENTION

The present invention realizes the prefixed aims since it is an anthropomorphic robotic hand comprising: a frame (00); a metacarpus (10), fastened to said frame (00) so to be able to rotate and translate along the axis (180) of a cylindrical joint; a thumb (11) fastened to said metacarpus (10) so to be able to rotate along the axis (140) of a rotoidal joint; an actuation mechanism of said thumb (11) and said metacarpus (10); at least two fingers fastened to said frame, which can be counterposed to said thumb and respective mechanism for their actuation, characterized in that the actuation mechanism of said thumb and said metacarpus comprises: a first sub-mechanism activated by a first motor (M2) integral to said thumb (11) and configured to move, by means of a worm screw (R2), a ring gear (T2) fastened so to mesh with respective teeth provided on said metacarpus (10), so that a rotation of said proximal phalanx to said metacarpus corresponds to a rotation of said ring (T2); a second submechanism actuated by a second motor (M3), integral to said frame (00) and configured to move, by means of a worm screw (R3), a ring (T3) that is configured to make said metacarpus (10) rotate to said axis (180) of said cylindrical joint, by means of a clutch (Cl).

### BRIEF DESCRIPTION OF THE DRAWINGS

FIGS. 1, 2 and 3 show kinematic schemes of the prosthesis according to the invention, with relative nomenclature;

FIG. 4 shows two overall views, from the back and palm, of a prosthesis with the thumb mechanism according to the invention installed, without the cover;

FIG. 5 shows the kinematic scheme of the thumb and metacarpus used in the prosthesis according to the invention;

FIG. 6 shows the actuator sub-mechanism of the thumb, actuated by the first motor;

FIGS. 7, 8 and 9 show various views of the submechanism of the metacarpus, actuated by the second motor;

FIG. 10 shows the three positions the thumb can take with respect to the palm;

FIGS. 11 to 14 show schematic views of the functioning of the Hall effect sensors provided in the device according to the invention, FIG. 15 shows some views illustrating the frame, the thumb and the realization of the glove for covering the thumb according to the invention; FIG. 16 shows some exemplificative views of the various grasps possible with the device according to the invention.

### DETAILED DESCRIPTION OF THE PREFERRED EMBODIMENTS

As it is shown in the appended figures, and without this limiting the aims of the invention, the prosthetic hand according to the present invention comprises 4 fingers, corresponding to the index finger (II), middle finger (III), ring finger (IV) and little finger (V) of the human hand, each comprising a middle-distal phalanx (mpn, mpni, mpiv and mpv, respectively for index finger, middle finger, ring finger and little finger), a proximal phalanx (ppu, ppm, ppiv and ppv, respectively for index finger, middle finger, ring finger and little finger) and an appendix (apn, apm, apiv and apv, respectively for index finger, middle finger, ring finger and little finger).

The distal and proximal phalanges are fastened to each other through an interphalangeal joint IP (IPu, IPm, IPiv and IPv, respectively for index finger, middle finger, ring finger and little finger), and the proximal phalanges are fastened to appendixes by the first one of two axes of rotation of a metacarpophalangeal joint (MCPu, MCPni, MCPiv and MCPv, respectively for index finger, middle finger, ring finger and little finger) in the flexion-extension plane; the appendixes are then fastened to a frame representing the palm (00) by the second one of the two axes of rotation of the same metacarpophalangeal joint in the abduction-adduction plane.

It is to be specified that the number of the phalanges of each finger can be also two, for example for the realization of particularly little hands, for which it is not technically possible or convenient to build mechanisms with three phalanges.

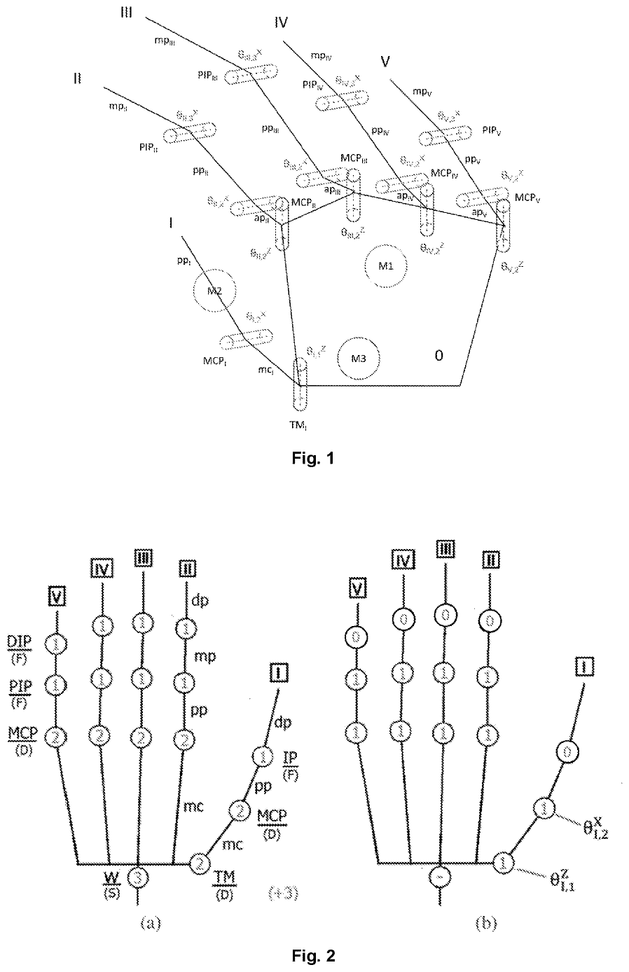

As it can be seen in the appended figures—see for example FIG. 1—the prosthesis according to the present invention comprises also a finger corresponding to the thumb (I), made up of a proximal-distal phalanx (ppi) fastened by means of a metacarpophalangeal joint (MCPi) to a metacarpus (mci), in turn fastened to the frame (00) by means of a trapezio-metacarpal joint (TMi).

Moreover, preferably, the hand according to the invention comprises four fingers corresponding to the index finger (II), middle finger (III), ring finger (IV) and little finger (V) of the human hand and a respective implementation mechanism. In particular, index finger and middle finger are configured to be able to be opposed to the thumb (I), to carry out various grasps needed to carry out daily life activities.

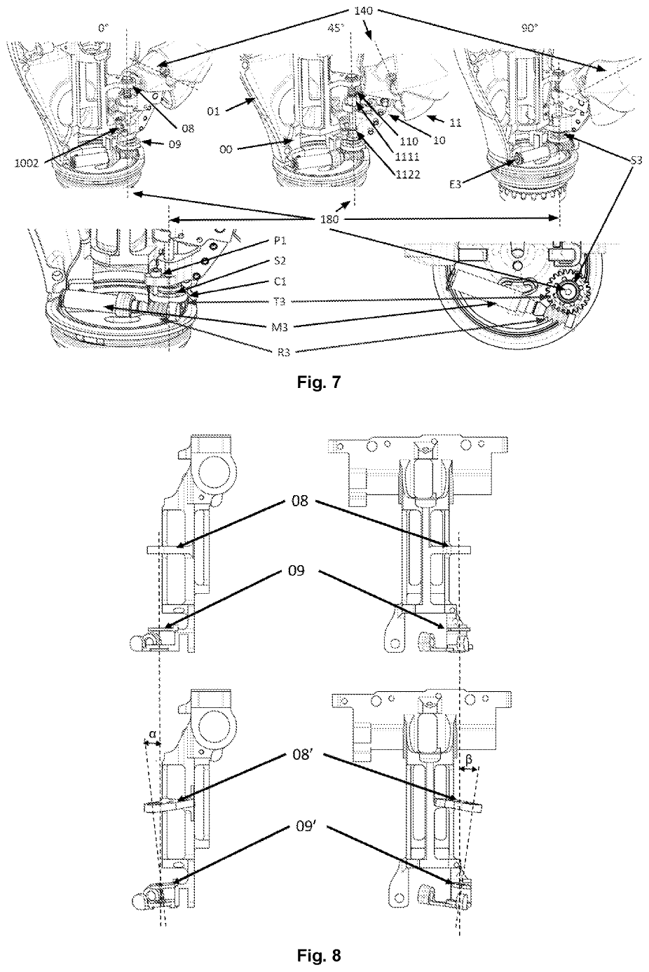

In the embodiment described in the following, the mechanism has 2 degrees of freedom, indicated by the variable 0i, jk, which refers to the rotation of the jth joint of the thumb (I) (j=1 for the trapezio-metacarpal joint TM; j=2 for the metacarpophalangeal joint MCP); the apex k refers instead to the angle interested by the movement; flexion-extension angle (k=X) or abduction-adduction angle (k=Z). Of these 2 degrees of freedom: one can be controlled actively and bidirectionally by means of a first motor (M2) positioned inside the proximal phalanx (ppi), corresponding to the flexion-extension movement of the joint MCPi, obtained with the rotoidal joint (0i, 2X) indicated in FIG. 7 with reference number (140); the other one can be controlled actively and bidirectionally by means of a second motor (M3) positioned in the palm (00), corresponding to the movement of abduction-adduction of the trapezio-metacarpal joint (TMi), obtained by the rotation allowed by the cylindrical joint (θ_{I,2}^{x}) indicated in FIG. 7 with the reference number (180); the cylindrical joint (180) allows also a vertical translation, which allows to uncouple the metacarpus temporarily from the second motor (M3), thus allowing to implement the degree of freedom relative to the rotation 0i,iz passively by means of the contralateral limb of the user.

Conveniently the device according to the invention comprises also a control electronic board configured to receive in input control signals (preferably but not limitingly by means of myoelectric sensors) and to control the motors provided as a function of said control signals. Moreover, preferably, the control board is configured to receive in input the signals coming from all the other sensors possibly provided on the device, such for example IMU sensors and position sensors, whose functioning will be explained in the following.

It is to be specified that with rotoidal joint it is intended a hinge coupling that allows only a relative rotation between the two interested elements and avoids relative translations, with cylindrical joint it is intended instead a coupling that allows both relative rotation and relative translation along the axis of rotation.

Moreover, two elastic elements (1111 and 1122) are provided which are interposed between the two elements in relative rotation of the trapezio-metacarpal joint (TMi) which allow rotations for little angles along the two axes perpendicular to the one of the joint, in order to cushion potential impacts received by the thumb (one of these two rotations corresponds in the human hand to the additional degree of freedom 0i, ix).

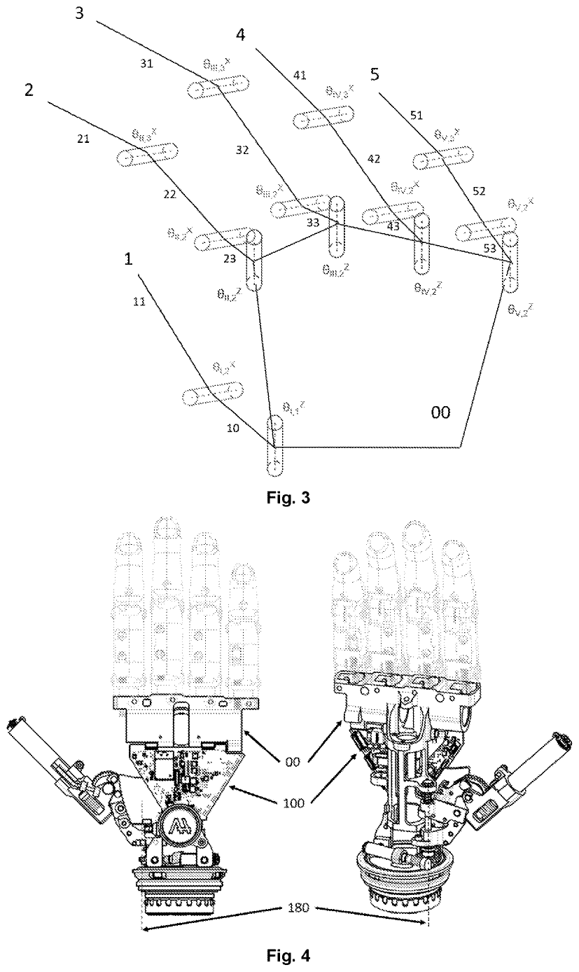

The frame is then preferably covered by a cover (01), which serves to protect the mechanism and said inner electronic board and to improve the appearance of the prosthesis, making it resemble the one of a human hand as accurately as possible. Due to the complexity of the mechanism, it is considered useful to introduce the criteria according to which nomenclature was assigned. The proximal-distal phalanx of the thumb is indicated with the number reference (11) and the metacarpus with the number reference (10). The axis of rotation x (flexion/extension) of the thumb between proximal phalanx and metacarpus is indicated with (140), while the axis of rotation of the metacarpus with respect to the palm is indicated with (180). It is now described a preferred embodiment of the mechanism, whose kinematic scheme is shown in FIG. 3.

It is to be specified that in FIG. 3 it is shown a right hand, observed by the back of the hand. Clearly, the same mechanism can be realized for a left hand. In the embodiment shown, it is reproduced the functioning of an anthropomorphic robotic hand, provided with four substantially aligned fingers, from the index finger to the little finger, and with an opposable finger thereto (thumb).

It is to be specified that with “aligned fingers”, in the present patent, it is intended the opposable fingers to the thumb and in the human hand corresponding to index finger, middle finger, ring finger and little finger. It is also to be specified that even if the device is described and shown with reference to four aligned fingers, the same can be also realized with only two aligned fingers, or with four aligned fingers, of which only two are movable and the others are fixed, without departing from the aims of the invention. In this way, cheaper devices in their realization without the metacarpus and thumb functioning being different from the one described and claimed here, are obtained.

The actuation mechanism of thumb and metacarpus is made up of two main sub-mechanisms, each one actuated by a different motor (M2, M3) or, exclusively in case of the second sub-mechanism, by the contralateral limb of the user (for example the left limb in case the prosthesis is used for the right hand).

As it is shown in FIG. 6, the proximal phalanx of the thumb (11) is made up of a metal element (112) integral to a cover in plastic (111). The motor (M2) is contained inside the proximal phalanx of the thumb (11) and its case is integral to the element (112).

The metacarpus (10) is conveniently made up of two sub-elements (101 and 102), the first one (101) being fastened with the cylindrical joint to the frame (00) at the axis of rotation (180) and the second one (102) being fastened with the rotoidal joint to the element (112) of the proximal phalanx of the thumb (11) at the axis of rotation (140). The two elements (101) and (102) are made integral to each other by means of a prismatic coupling, which is blocked in position by means of a bolt at the axis (160).

At the axis of rotation (140) between the two elements (102) of the metacarpus (10) and (112) of the proximal phalanx of the thumb (11) and contained inside the element (112) it is also provided a ring gear (T2), idle to the axis (140) between the two elements. On one of the two sides the ring (T2) is provided with teeth meshing with as many teeth positioned on the second element (102) of the metacarpus (10). On the other side of the ring (T2), between ring and element (102), it is instead provided a Belleville washer (Sl).

The motor (M2) meshes with the ring (T2) by means of a worm screw (R2), so the proximal phalanx of the thumb (11) rotates to the metacarpus (10) around the axis (140) between a first and a second position. In a preferred embodiment the maximum rotation allowed between said first and second position is 55°.

When the thumb is in contact with the object, or it is at its limit stop, the movement of the proximal phalanx is stopped, and the motor (M2) is consequently forced to stop. By controlling the current of the motor, the maximum torque exerted is determined, and so, the maximum torque transmitted to the thumb determining the grasp force. It is also possible to control the speed of the motor with an encoder (E2) to control the speed the grasp is carried out with.

In case the thumb is subjected to undesired outer stresses, the Belleville washer (Sl) is compressed, thus allowing the ring (T2) to translate along the axis (140), disengaging itself temporarily from the second element (102) of the metacarpus. In this way, the thumb rotates freely, absorbing the outer stresses, so its elements are not damaged. The rigidity of the spring (Sl) and the profile of the teeth on the ring (T2) and on the second element (102) of the metacarpus are clearly fixed so to make the torque needed to obtain such disengagement higher than the one applied by the motor (M2) to make the thumb (11) rotate, and lower than the one which would damage the elements of the thumb.

So, in this way, the assembly is protected from undesired stresses with tend to flex or extend the thumb relative to the metacarpus, thus increasing the compliance of the device and creating a “buffer zone” between thumb and metacarpus, that guarantees a longer duration of the elements used. Such measures are absent in patent as WO2021250233A1 and WO2022038506A1, while WO2020065266A1 uses a system based on collapsible rods, which allows a forced closing of the thumb caused by outer stresses without the mechanism being damaged but does not protect the thumb from stresses tending to open it. So, such devices are more subjected to breaking upon outer stresses, which are the most frequent causes of breaking in this kind of devices. U.S. Ser. No. 11/351,042B2, instead, provides one or more configurations in which the thumb comprises a coupling system intending to protect it from such uncontrolled stresses. Such system comprises driven elements, blocking elements and compression elements, as in the case of the present patent, but the substantial difference is the constructive complexity. Specifically, the device according to the present invention is characterized by simple construction since the elements constituting the protection system from impacts are few, unlike the known devices in which the many elements make more expensive the assembly and increase the risk of mal functioning of the system due to breaking of the elements. For example, in the case in which it is needed to disassemble the mechanism to replace an element: in the device according to the present invention, it is sufficient to remove the shaft provided at the axis (180) to access to the elements configuring the system; in the case of U.S. Ser. No. 11/351,042B2 it is needed to disassemble all the elements constituting the joint from their own seat. The elements 103 and 104, integral to the metacarpus, protect the mechanism from the outside. Preferably, the phalanx of the thumb comprises, at the fingertip, a pressure sensor which is used to control the behavior of the motor of the thumb and other fingers.

In particular, since the patient using a prosthesis has not clearly proprioception G of the limb and of when the object grasped is stable, it is possible that to happen. For example, if a glass is grasped by means of the prosthesis and water is filled in such glass in the following, while the water quantity in the glass increases (and so its weight) it can happen that the prosthesis has not the strength sufficient to hold it, so the glass slides and falls. The function of the pressure sensor is to determine if this is happening. To such aim, the pressure sensor is configured to measure the pressure provided between fingertip and grasped object, and the control board is configured to increase the torque applied in case a pressure reduction is detected, so that if the object slides and the sensor measures a pressure reduction, the device closes autonomously, without the need of an active control by the patient.

In particular, the control electronic board is configured to detect continuously the pressure exerted by the fingertip on the object following a grasping action, and if such pressure is reduced without the patient has carried out opening operations of the device, to control the hand to increase the closing strength up to when the sensor measures a value of pressure again at least equal to the one measured before the undesired reduction. As it is shown in FIG. 7, the motor (M3) is fixed to the frame (00) and meshes with the ring (T3) by means of a worm screw (R3). Such ring (T3) is provided, on the side surface, with teeth meshing with respective teeth positioned on the side surface of a clutch (Cl), free to rotate on the same axis of rotation (180). As it is shown in figure (7), said clutch (Cl) is provided with holes along the whole circumference, with axes parallel to the ones of rotation of the clutch itself, and arranged preferably but not limitingly at 45° to each other, in which a pin (PI) coming out from the metacarpus (10) is engaged, thus making the two elements integral to the rotation along the axis of rotation (180). The metacarpus (10) is fastened with two cylindrical joints to a shaft integral to the frame at the axis of rotation (180). Between shaft and metacarpus, at such two hinges, elastic cylinders (1111 and 1122) are introduced, which allow rotations for little angles in the two planes perpendicular to the one of the joint, in order to cushion potential impacts received by the thumb.

So, the motor M3 can put into rotation the ring (T3), and so the clutch (Cl), thus causing the movement of rotation of the metacarpus (00) around the axis (180). In this way, the thumb is moved from the side position to the opposition one to the other fingers and vice versa. Moreover, in such configuration, the mechanism is not back drivable, so the worm screw prevents the thumb from being able to rotate due to outer stresses, thus making the grasp very strong.

Some prostheses of upper limb known at the state of the art implement mechanisms trying to carry out the same functions, as for example the device described in US2018036145A1, which sometimes is however difficult to be controlled: the user, in fact, has to be able to control both the fingers flexion and the rotation of the thumb and since he usually has only two control inputs (the two surface electromyographic sensors) which he uses for the movement of fingers “opening” and “closing”, to be able to control also the movement of rotation he is forced to carry out complex muscle contraction patterns, with the result of a control, which is often not intuitive, and long learning times.

Other devices available on the market allow instead the user to move the rotation of the thumb passively, with the contralateral limb or by using an outer counter-surface. But, in order to make it possible that the action of the user allowing the passive rotation of the thumb does not require an excessive effort by him, thus resulting uncomfortable, such systems use mechanisms based on friction or on partial meshing between metacarpus and frame, which do not fasten such elements firmly. So, such mechanisms are often subjected to unvoluntary actuation due to undesired outer stresses, with the consequent undesired rotation of the thumb, resulting in a less firm and less reliable grasp in the daily life.

The mechanism described in WO2020065261 allows instead the active rotation by means of muscle control, and a “false” passive rotation by means of a button provided near the thumb, which the user can actuate with the contralateral limb and which causes the actuation of the motor. But such mechanism does not allow the rotation of the thumb when the hand is not fed, neither it allows to position the metacarpus in an intermediate position between the two limit stops corresponding to the thumb in side position (0°) and thumb in opposition to the index-little fingers (90°). In the case of the device according to the present invention, instead, the user can disengage the metacarpus (10) from the clutch (CI) by means of a vertical translation of the metacarpus (10) upwards, so to bring the pin (PI) of the metacarpus (10) outside the hole of the clutch (Cl) in which it is engaged. At this point, the user, while maintaining the metacarpus (10) lifted, can rotate it of 45° or 90°, and then release it. The linear spring (110) provided between metacarpus and frame brings the metacarpus back in position, engaging the pin (PI) again in the new selected hole of the clutch (Cl).

In order to make this operation easier, the holes on the clutch (Cl) are provided with flares in the portion facing the metacarpus, in order to guide the pin (PI) of the metacarpus (10) towards the holes even if the user does not carry out the release exactly at the same. The rotation limit stops are realized by means of bumps provided on frame and metacarpus, which limit the maximum relative rotation between the two elements to 90°.

So, the just described mechanism overcomes the limits of the systems currently available on the market, allowing to carry out the rotation of the thumb both actively and continuously, by means of the motor (M3), both passively and discretely, with determined steps from the angular distance of the holes provided along the whole circumference of the clutch (Cl), by means of the contralateral limb of the user, thus allowing a high easiness of usage and a firm grasp at the same time. The movement can be also realized with prosthesis not fed, thus increasing the safety in dangerous situations in which it is needed to release the grasp.

Moreover, a Belleville washer (S2) is provided between clutch (Cl) and frame. When the thumb is subjected to high rotational stresses, such spring is compressed, allowing the clutch (Cl) to translate upwards along the axis (180), disengaging itself from the ring (T3). In this way, the metacarpus rotates freely, absorbing the outer stresses, so the elements of the thumb are not damaged. The rigidity of the spring (S2) and the profile of the teeth on ring (T3) and clutch (CI) are clearly fixed so to make the torque needed to obtain the disengagement higher than the one carried out by the motor (M3) to rotate the metacarpus (10), and lower than the one which would damage the elements of the thumb.

Between metacarpus and frame it is also provided a second Belleville washer (S3), which has the function to absorb the impacts that tend to bring the metacarpus (10) downwards. For the impacts that tend instead to bring the metacarpus (10) upwards, the same function is carried out by the linear spring (110).

So, in this way, the assembly is protected both from undesired stresses tending to translate the thumb upwards or downwards, and stresses tending to rotate it towards the inside or outside of the palm, increasing the compliance of the device and creating a “buffer zone” between thumb and palm that guarantees a longer duration of the elements used. Such measures are absent in patents as WO2021250233 and WO2022038506A1, which do not protect the thumb from stresses tending to rotate the metacarpus towards the inside or the outside of the palm and to move it upwards or downwards. The system provided in U.S. Ser. No. 11/351,042B2 gives instead the function of absorbing such stresses mainly to the material used for the user support, able to be deformed elastically. So, upon an impact downwards or upwards, the support tends to rotate and with it the actuator as well, actuating a mechanism whereby two blocking surfaces, on palm and thumb respectively, are engaged to each other, thus avoiding another relative rotation. On the contrary, in the device according to the present invention, the elements absorbing the impacts that tend to rotate the thumb are linear springs (110) and Belleville washers (S3), and the support of the actuator (and so of the actuator itself) is not subjected to rotations after outer stress, since such support is fixed, since it is constituted by flanges of the frame (00). So, the actuator has not to follow directly the direction of the outer stress, gaining in stability and less direct exposition to stresses.

Also DE102021132277B3 provides a system for protecting the thumb from outer stresses, which however has substantial differences concerning the mechanical structure in comparison to the device object of the present invention: in the present patent supporting elements, intended to support the elements of the joint, are not provided but it is the frame (00) itself that carries out this function by means of the addition of flanges (08, higher, and 09, lower) visible in FIG. 7. Moreover, for the transmission of the movement and to give rigidity to the torsion, it is not needed to introduce other elements with outer and/or inner teeth, since it is the same pin which blocks the rotation of the metacarpus, to serve as connection for the higher portion of the metacarpus and so to rotate the thumb. Such pin is realized and designed in sufficiently rigid material to carry out this function.

When the metacarpus (10) goes in contact with the grasped object (or arrives to limit stop) the motor (M3) is forced to stop.

By controlling the current of the motor, the maximum torque exerted is determined, and so, the maximum torque transmitted to the metacarpus determining the grasp force. It is also possible to control the speed of the motor with an encoder (E3) to control the speed the metacarpus is moved with and the grasp force.

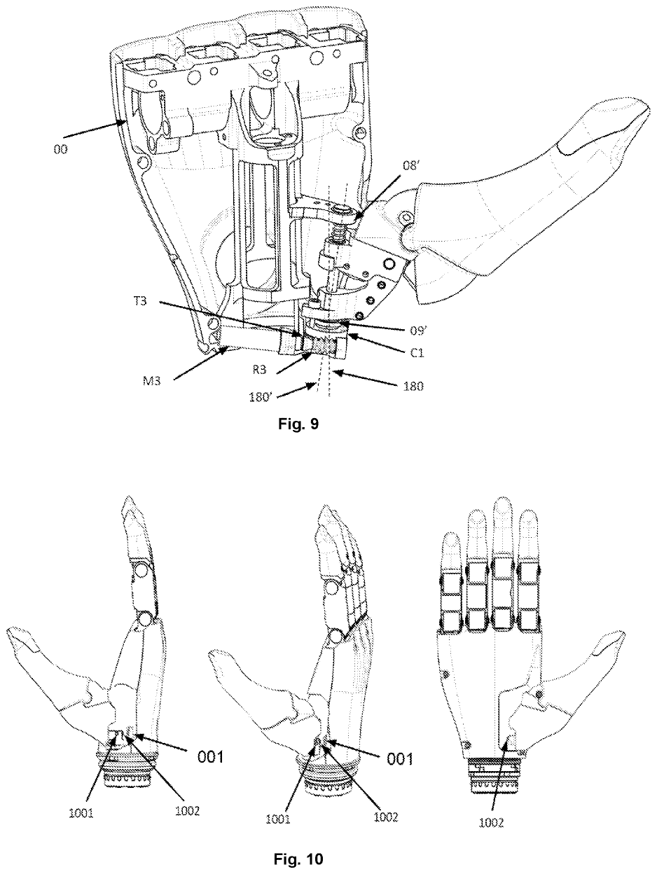

In the device according to the present invention, it is not needed that the abduction-adduction axis of the trapezio-metacarpal joint (180) is parallel to the axis of the forearm, as it is shown in FIG. 4, but it can be inclined to the axis of the forearm, with the aim of making the prosthesis more similar to a human hand. Such inclination can be provided along both the sagittal and coronal planes. Such inclination is obtained by rotating both the connection flanges (08 and 09) of the frame (00) to the metacarpus (10) according to two directions, defined by angles a and 0 (FIG. 8) respectively. So, the axis of the metacarpus (180) is inclined firstly according to the angle a and then according to the angle 0; also the flanges of the frame (00) are inclined analogously, becoming (08′) and (09′) respectively. There are obtained two flanges parallel to each other which allow to assemble metacarpus and so thumb, while maintaining the geometry of these elements unvaried. In this way, the only element to be modified is the frame (99), FIG. 9.

In fact, unlike what happens in the Patent 102016000120646, such variation can be provided without modifying any element, if not the frame (00), greatly simplifying the construction of other mechanism variants.

In the case of the two sub-mechanisms described, the usage of a reduction unit with worm screw, in addition to provide a very compact way to carry out the reduction of speed and the increase of motor torque, is also a not invertible mechanism: by acting on thumb and metacarpus, such elements cannot be reopened because it is impossible to transmit the movement from the rings (R2, R3) to the motors (M2) and (M3), respectively.

Once the full closing of thumb and metacarpus is reached, it is so possible to turn off the motors (thus saving battery charge), since the grasp remains stable anyway, because the fingers cannot be reopened passively, unless using possible safety devices in case of danger.

Moreover, the control of the grasp force can be carried out by controlling the motor current, and this can be carried out simply in many ways.

For example, the current can be controlled as a function of the number of signals read by the control electrodes in a determined time interval (for example: 1 signal in 1 second for a soft grasp, 2 signals in 1 second for a middle grasp, 3 signals in 1 second for a power grasp).

As an alternative, the current absorbed by the motor (and so the torque exerted by the same which determines the grasp force) can be controlled as a function of the intensity of the signals detected by the electrodes, correlating it in directly proportional way or according to other calibration law to be defined.

The compactness of the mechanism allows to position inside the manipulator also the control board (100), as it is shown in FIG. 4, so the manipulator acquires another degree of modularity. This is particularly useful in case this has to be used by subjects with different levels of amputation: as a way of example, for a transradial amputation a “wrist” module or a “forearm” module can be added to the module of the manipulator, while in case of amputation under the wrist the manipulator module can be used singularly.

It is also clear, from the previous description of the mechanism, that the functioning is absolutely analogous both for the closing movement of the hand and for the opening movement of the same. In fact, all the connections used are bidirectional (gears) and unidirectional elements are not used (tendons, tension rods . . . ) which have also the limit of being not able to exert great forces and to have higher inner frictions.

Moreover, due to the exclusive usage of gears transmissions, the mechanism is isotropic, so independently of the kinematic position of the finger they exert a constant force, thus guaranteeing constant performances in the whole work space.

It is clear that the kinematic scheme shown is to be intended exemplificative and not limiting the aims of the invention. It is to be specified, as a way of example, that the dimension of the limb can be modified by changing, according to the needs, the module of the gears, the number of their teeth or the dimension of the motors.

As yet described, the device according to the present invention allows to realize the rotation of the metacarpus with respect to the palm, both actively and passively. In particular, while the active rotation is continuous, the passive rotation allows to position the thumb in three positions, shown in FIG. 10: lateral position (c), with the thumb substantially coplanar to the palm;
tridigital position (a), with thumb opposite to the index and middle finger, and so forming an angle of about 90° with respect to the palm; neutral position (b), intermediate between the two previous ones.

The device is configured to coordinate the closing movement of the fingers and of the thumb by means of the control board (100), in order to realize all the main grasps needed in the daily life.

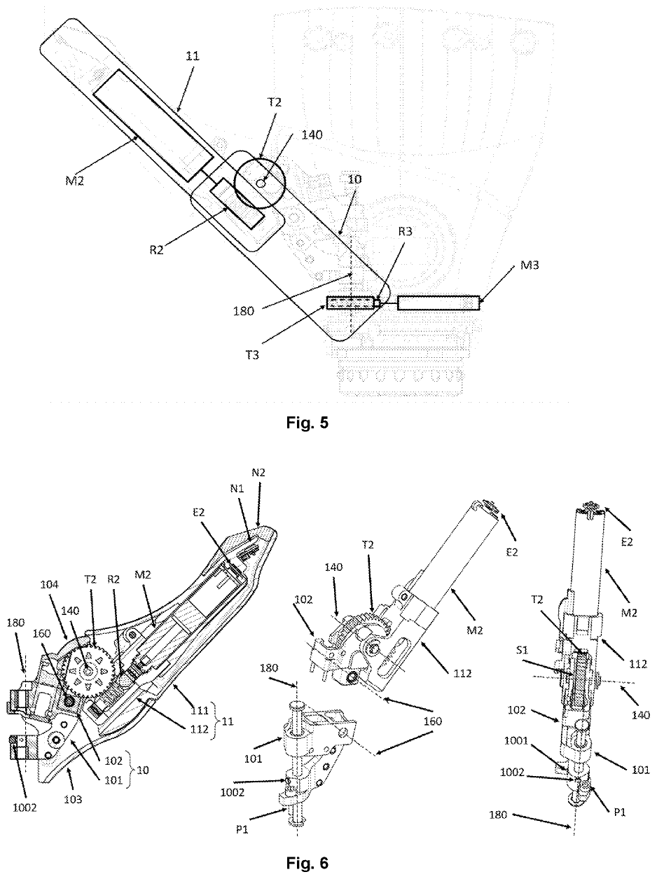

Preferably, in fact, the device comprises an inertial unit configured to acquire the orientation angle of the robotic hand with respect to the horizontal direction, and it is configured to carry out, as a function of the signals detected by the electromyographic sensor, the position sensor of the thumb and the inertial unit, the following grasps:
1. Tridigital grasp (FIG. 16-1): when the thumb is in tridigital position its fingertip closes against the fingertip of index and middle finger. This grasp is ideal to grasp little objects.
2. With the same configuration of the thumb and hand, but in case larger objects are grasped, a power grasp (FIG. 16-2) is automatically carried out, wherein also the fourth and fifth finger of the robotic hand are in contact with the grasped object.
3. When the thumb is in tridigital position and the hand is facing downwards (between −80° and −90° with respect to the horizontal plane) or upwards (between +80° and +90° with respect to the horizonal plane), the thumb closes after the other fingers, blocking index and middle finger in position. In this way, a strong hook grasp is obtained, useful for heavy objects if the hand is facing downwards (FIG. 16-3) or to grasp for example a handle in a bus if the hand is facing upwards.
4. “Handshake” grasp (FIG. 16-4): when the thumb is in neutral position and the hand is in an interval between +90° and −80° with respect to the horizontal plane, all the fingers close together naturally, in an ideal grasp for a handshake and similar actions.
5. Grasp for typing (FIG. 16-5): when the thumb is in neutral position the hand is facing downwards (between −80° and −90° with respect to the horizontal plane), the thumb closes before the other fingers and against the side of the middle finger, thus offering a support base for the index finger, which can be used to type on a keyboard.
6. Lateral grasp (FIG. 16-6): when the thumb is in side position and the hand is completely closed, the opening and closing movement controls only the opening and the closing of the thumb, without involving the other fingers, that so offer a stable support for keys, credit cards or smartphone. To obtain the control of the whole hand again it is needed to reopen the thumb wholly.
7. Rest position (FIG. 16-70: 3 seconds after that the hand is completely open, it goes automatically in a more natural and relaxed position.

The actuation angular values of the various grasps can be modified by the orthopedic technician by means of a suitable software to which the hand is connected via Bluetooth. Such software allows also to actuate and disactivate the described functions, to provide the patient with the maximum customization possible for the device functioning.

| | | Thumb position | | |
|---|---|---|---|---|
| | | Opposition | Intermediate | Lateral |
| Hand angle | Forward | 1) tridigital | 4) handshake | 6) lateral |
| with | (+80° ÷ −80°) | grasp | grasp | grasp |
| respect to | | 2) power grasp | | |
| horizontal | Upwards | 3) hook grasp | | |
| plane | (+80° ÷ +90°) | | | |
| | Downwards | | 5. grasp for | |
| | (−80° ÷ −90°) | | typing | |

According to a preferred embodiment, the prosthetic hand according to the present invention comprises an actuation mechanism of the thumb of the just described type and is provided with a control system configured so to not cause the user to carry out complex muscle contraction patterns to carry out all the main kinds of grasps needed in a standard day.

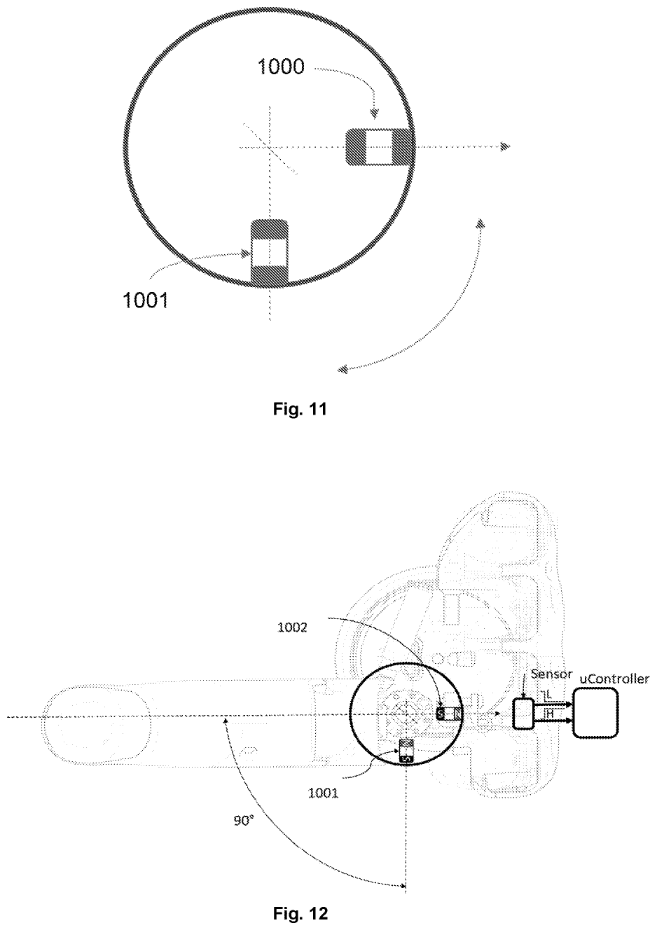

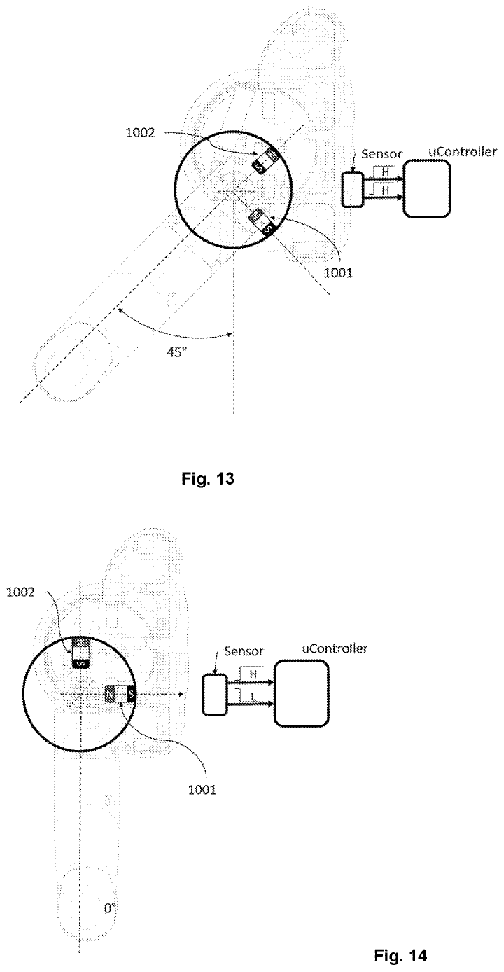

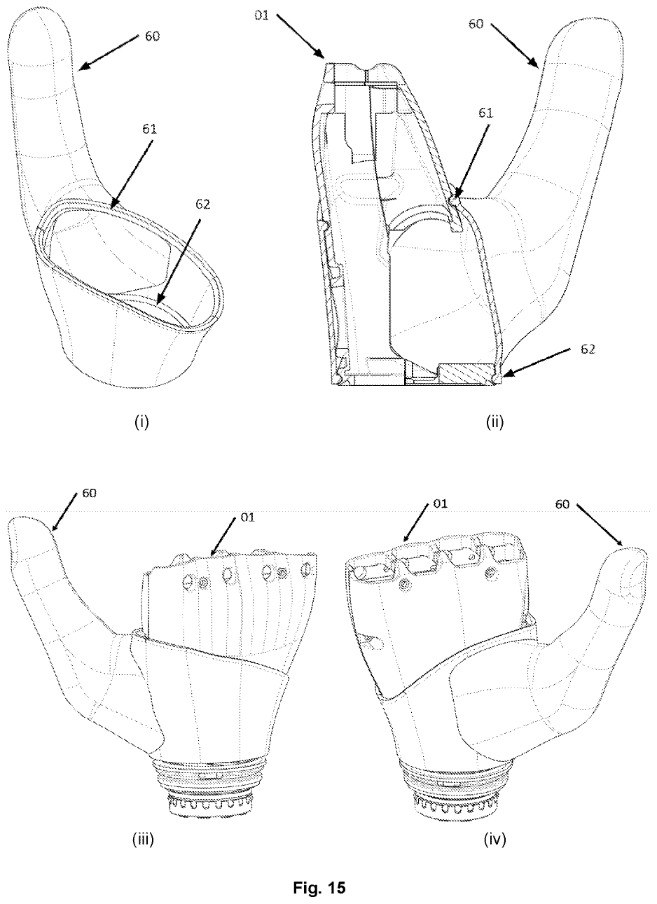

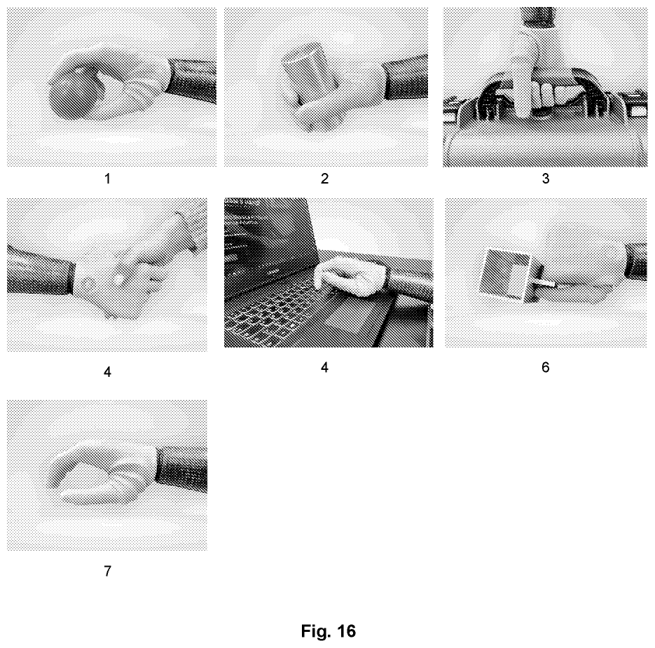

With reference to the appended FIGS. 11 to 14, it is to be specified that, according to a preferred embodiment, the device according to the present invention comprises a thumb position sensor, able to determine the angle of rotation of the metacarpus to the frame. In particular, the thumb position sensor comprises: a Hall effect sensor (001) integral to the frame (00) and two magnets (1001, 1002) integral to the metacarpus (10).

The Hall effect sensor is provided with two outputs: the first one is actuated if it detects the presence of a magnetic field “going out” from the magnet generating it and the second one is actuated if the sensor detects a magnetic field “going in” the magnet generating it.

The sensor detects the variation of the magnetic field, generating a low logic level on the respective pin. So, in presence of a magnetic field, the sensor brings the pin to a low logic level, while, when the magnetic field stops acting, the respective pin is brought back to a high logic level.

By positioning the two magnets (1001, 1002) on the metacarpus, oriented opposite to each other, so to generate two magnetic fields with direction opposite to each other, it is possible to determine three positions.

The two magnets are oriented opposite to each other, so to generate two magnetic fields of opposite direction to each other.

As a function of the position of the thumb with respect to palm, as it is shown in FIGS. 12, 13 and 14, the two outputs of the Hall effect sensor can be: the first one in a low logic state and the second one in a high logic state, and the sensor will recognize the rotation of 90° with respect to the palm: both in high logic state, and the sensor will recognize the rotation of 45° with respect to the palm; the first one in a high logic state and the second one in a low logic state, and the sensor will recognize the rotation of 0° with respect to the palm.

Considering what is known at the state of the art, for example described in EP2125091 or in DE102021132277, the presence of an inertial sensor allows to determine the kind of grasp not only as a function of the thumb position, but also as a function of the hand orientation, thus making the actions, which can be carried out by the robotic hand, more similar to the ones carried out by human hands.

It is also to be noticed that the shaft at the axis (160) is conveniently made up of a bolt, which can be screwed and unscrewed without removing other elements of the hand, so to remove and replace the thumb easily in case of breaking.

The protheses of higher limb are usually not designed to be repaired directly on site by orthopedic technicians, since they have a very complex design. So, the orthopedic technicians have to send the device in the manufacturer's headquarters for any failure, and this causes long waiting times for the users and high costs for the manufacturer, who is compelled to send a temporaneous substitute device to the user, while the device is repaired. The few prostheses provided with replaceable fingers not only do not usually allow to replace the thumb but they are also not waterproof, since it is technically very difficult to design a seal between fingers and palms which functions in a repeatable way after the orthopedic technicians disassemble and replace fingers.

The designed mechanism overcomes this limit by means of a partial glove (60) covering the thumb, the metacarpus and a portion of the cover of the hand and is sealed on the cover thanks to suitable seats in which specific bumps provided on the glove (61, 62) are introduced (FIG. 15). In particular, the glove seal is guaranteed by the compression of such bumps in the cover seats, obtained by realizing such bumps with slightly smaller diameter and slightly greater thickness than the seats provided on the cover.

1. An anthropomorphic robotic hand, comprising:
a frame (00);
a metacarpus (10), fastened to said frame (00) so to be able to rotate and translate along the axis of a cylindrical joint (180);
a thumb (11) fastened to said metacarpus (10) so to be able to rotate along the axis of a rotoidal joint (140);
an actuation mechanism of said thumb (11) and said metacarpus (10);
at least two fingers fastened to said frame, which can be counterposed to said thumb and respective mechanism for their actuation; characterized in that the actuation mechanism of said thumb and said metacarpus comprises:
a first sub-mechanism activated by a first motor (M2) integral to said thumb (11) and configured to move, by means of a worm screw (R2), a ring gear (T2) fastened so to mesh with respective teeth provided on said metacarpus (10), so that a rotation of said proximal phalanx to said metacarpus corresponds to a rotation of said ring (T2);
a second sub-mechanism actuated by a second motor (M3), integral to said frame (00) and configured to move, by means of a worm screw (R3), a ring (T3) that is configured to make said metacarpus (10) rotate to said axis of cylindrical joint (180), by means of a clutch (Cl).

2. The anthropomorphic robotic hand according to claim 1, wherein said ring (T3) is provided, on the side surface, with a plurality of teeth meshing with respective teeth positioned on the side surface of a clutch (Cl), free to rotate on the same axis of cylindrical joint (180).

3. The anthropomorphic robotic hand according to claim 2, wherein said mechanism comprises also a Belleville washer (Sl), configured to be compressed under the action of outer stresses thus allowing the ring (T2) to translate along the axis of rotoidal joint (140), disengaging itself temporarily from said respective teeth provided on said metacarpus (10).

4. The anthropomorphic robotic hand according to claim 3, wherein the rigidity of said spring (Sl) and the profile of the teeth on ring (T2) and on metacarpus are clearly fixed so to make the torque needed to obtain such disengagement higher than the one applied by the motor (M2) to make the thumb (11) rotate.

5. The anthropomorphic robotic hand according to claim 2, wherein said clutch (Cl) is provided with a plurality of holes having axes parallel to the ones of rotation of the clutch itself, configured to receive a pin (PI) integral to said metacarpus (10) making the two elements integral to the rotation along the axis of cylindrical joint (180), and to release said pin (PI) upon a translation of said metacarpus along said axis of cylindrical joint (180), making the rotation of the metacarpus free with respect to said frame (00), said mechanism comprising also a spring (110) positioned between metacarpus (10) and frame (00) and configured to push said metacarpus (10) along said axis of the cylindrical joint towards said clutch (Cl).

6. The anthropomorphic robotic hand according to claim 5, wherein said holes provided on the clutch (Cl) are provided, in the portion facing said metacarpus, with flares configured to make the introduction of said pin (PI) easier.

7. The anthropomorphic robotic hand according to claim 1, wherein said second sub-mechanism can be actuated manually, by making the metacarpus translate so that said pin (PI) does not engage the holes provided on said clutch.

8. The anthropomorphic robotic hand according to claim 1, further comprising a Belleville washer (S2) positioned between said clutch (Cl) and said frame (00), configured to be compressed upon high rotational stresses of said thumb (11), thus allowing the clutch (Cl) to translate upwards along the axis of cylindrical joint (180), disengaging itself from the ring (T3).

9. The anthropomorphic robotic hand according to claim 8, wherein the rigidity of said Belleville washer (S2) is such that the torque needed to obtain such disengaging is higher than the one applied by said motor (M3) to make said metacarpus (10) rotate.

10. The anthropomorphic robotic hand according to claim 1, wherein said metacarpus (10) is fastened with two cylindrical joints to a shaft integral to the frame at the axis of cylindrical joint (180), between said shaft and said metacarpus being interposed, at such two hinges, elastic cylinders (1111 and 1122), which allow rotations for little angles.

11. The anthropomorphic robotic hand according to claim 1, wherein between said metacarpus (10) and said frame (00) a second Belleville washer (S3) is provided, which has the function to absorb the impacts tending to bring the metacarpus (10) downwards.

12. The anthropomorphic robotic hand according to claim 1, further comprising a control electronic board configured to receive in input control signals by the user and to control said motors (M2, M3) as a function of said control signals.

13. The anthropomorphic robotic hand according to claim 1, further comprising a pressure sensor arranged on the fingertip of the thumb (11) and wherein said control board is also configured to receive in input the signals coming from said pressure sensor and to increase the torque applied by said first motor (M2) in case, in absence of an opening operation of the hand by the user, said pressure sensor detects a reduction of the pressure exerted.

14. The anthropomorphic robotic hand according to claim 1, wherein said axis of said cylindrical joint (180) is inclined with respect to the axis of the forearm, and in that said inclination is obtained by rotating both the connection flanges (08 and 09) of the frame (00) to the metacarpus (10).

15. The anthropomorphic robotic hand according to claim 1, wherein said thumb (11) is configured to take, with respect to said frame:
a lateral position (c), in which said thumb (11) is substantially coplanar to said palm (00);
a tridigital position (a), in which said thumb forms an angle of about 90° with respect to said palm (00);
a neutral position (b), intermediate between the two previous ones.

16. The anthropomorphic robotic hand according to claim 1, further comprising a thumb position sensor, configured to detect the angle formed by the thumb with respect to said frame and an inertial unit configured to detect the angle formed in the space by said robotic hand.

17. The anthropomorphic robotic hand according to claim 16, wherein said thumb position sensor comprises: a Hall effect sensor (001) integral to the frame (00) and two magnets (1001, 1002) integral to the metacarpus (10).

18. The anthropomorphic robotic hand according to claim 17, wherein said robotic hand is configured to change the kind of grasp to be carried out as a function of the signals detected by the electromyographic sensor, the thumb position sensor and the inertial unit.

19. The anthropomorphic robotic hand according to claim 1, wherein said frame (00) is covered by a cover (01) configured to protect the mechanism and said inner electronic board, and said robotic hand comprises also a partial glove (60) configured to cover the thumb and the metacarpus and to exert a seal on said cover.
